(19) United States
(12) Patent Application Publication
(10) Pub. No.: US 2004/0241124 A1
(43) Pub. Date: Dec. 2, 2004

(54) Method for stabilizing an aqueous dispersion of an oil in silicone oil emulsion

(76) Inventors: Helene Lannibois-Drean, Charenton Le Pont (FR); Jean-Marc Ricca, 10/01 Orchard Bel-Air (FR); Mathias Destarac, Paris (FR); Philippe Olier, Lyon (FR)

Correspondence Address: Jean-Louis Seugnet, Intellectual Property Dept, Rhodia, 259 Prospect Plains Road, CN 7500, Cranbury, NJ, 08512-7500, US

(21) Appl. No.: 10/481,166

(22) PCT Filed: Jul. 1, 2002
(86) PCT No.: PCT/FR02/02279

(30) Foreign Application Priority Data
Jun. 29, 2001 (FR) 01 08652

(51) Int. Cl. A61K 7/06; A61K 7/11

(52) U.S. Cl. 424/70.12

(57) ABSTRACT

The invention relates to a method for stabilizing an aqueous formulation, comprising up to 40% by weight of one or several surfactants and at least one organic phase chosen from organic or mineral oils/greases/waxes, the products of alchoholysis of said oils/greases/waxes, essential oils, the mono-, di- and tri-glycerides, the fatty acids, the esters of said acids and the alcohols, individually or combined. According to said method, an emulsion is introduced into the aqueous composition, the continuous phase of which is at least one silicone, the dispersed phase is the organic phase as above stabilized by at least one graft or block copolymer, one fraction of which is soluble in the dispersed phase, the other soluble in the continuous phase, whereby the fraction soluble in the continuous phase is greater than the fraction soluble in the dispersed phase.

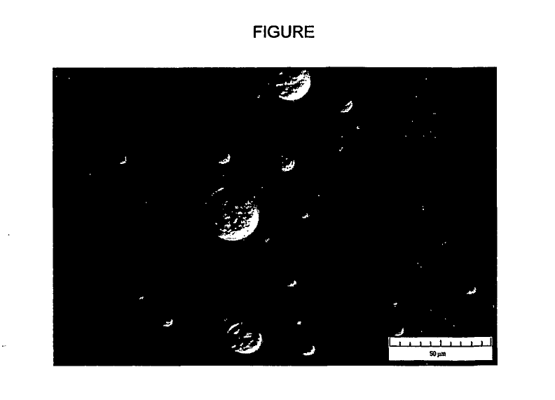

The present invention relates to a method of stabilizing a formulation containing up to about 40% by weight of surfactants and at least one organic or inorganic oil and/or a derivative.

More and more often consumers are being proposed a plurality of simultaneous and/or successive effects with a single formulation. Such performance is obtained by combining two or more active substances in the formulation.

Although this does not pose any major difficulty when the active substances are compatible with one another and with the formulation into which they are introduced, this is not the case when the desire is to incorporate compounds such as, in particular, organic and/or inorganic oils/waxes/fats into aqueous formulations with possibly high surfactant contents.

In fact, if these compounds are added to this type of formulation, the formulations may be observed to suffer destabilization, such as, for example, a destructuring of the surfactant phases, where present.

An additional difficulty exists which is encountered with the use of certain of the aforementioned oils/waxes/fats and which is linked to their relative sensitivity toward oxidation. Without special protection, the ethylenic unsaturations they contain are oxidized with greater or less rapidity by certain components of the formulation into which it is introduced. The consequences of this oxidation are often adverse or even prohibitory to the subsequent use of these formulations. The latter may, indeed, have their performance impaired, for example, by the deactivation of the oil/wax/fat. Similarly, the appearance of the formulations, such as the odor and the coloring, among other factors, may be undesirably modified.

When one is confronted with this kind of situation, one of the solutions is to protect the substance in question in order to isolate it from the medium into which it is introduced. However, the methods employed are not always effective and lasting.

The present invention accordingly provides a method of stabilizing an aqueous formulation comprising one or more surfactants with a surfactant content of up to 40% by weight of the formulation and comprising at least one organic phase selected from organic or inorganic oils/fats/waxes, products obtained from the alcoholysis of these oils/fats/waxes, essential oils, mono-, di-, and tri-glycerides, fatty acids, the esters of such acids, and alcohols, alone or in combination; wherein there is introduced into the aqueous formulation an emulsion:

whose continuous phase is at least one silicone, the disperse phase being the aforementioned organic phase;

and which is stabilized by at least one comb or block copolymer of which one fraction is soluble in the disperse phase and the other in the continuous phase, the fraction soluble in the continuous phase being greater than the fraction soluble in the disperse phase.

The method according to the invention, by isolating/protecting an organic and/or inorganic oil/wax/fat and/or derivative from the formulation, makes it possible to avoid any degradation of said compound, or any destabilization of the formulation, in particular of any structured surfactant phases.

Accordingly, the stabilized formulations according to the invention retain the properties provided by the oils/waxes/fats and also their characteristics such as the coloring, odor, etc.

It should be noted—and this constitutes another interesting characteristic of the present invention—that it is possible to observe a retardation of the effect exerted by the organic phase dispersed in the silicone. To cite just one example, the invention allows the combination, in a single formulation, of, on the one hand, a silicone, whose conditioning effect on the hair may be exerted at the time of application, and, on the other hand, an oil/wax/fat, whose nutrient/reparative effect on the hair will be obtained only after said oil has diffused through the silicone barrier deposited on the hair.

It should be noted that the method according to the invention is also a means of delivering said oils/waxes/fats.

Finally, the sensorial effect resulting from the application of a formulation comprising the emulsion or one of the dispersions according to the invention is modified favorably, as is the texture of the formulation.

Still other advantages and characteristics of the invention will appear more clearly on reading the description and the example which now follow.

Moreover, the figure represents an optical microscope view of a multiple emulsion diluted in a surfactant-rich aqueous phase.

In order to simplify the account, the emulsion will be described first of all.

As indicated above, the emulsion is such that the organic phase, which represents the dispersed phase, is selected from organic or inorganic oils/fats/waxes, products obtained from the alcoholysis of these oils/fats/waxes, essential oils, mono-, di- and tri-glycerides, fatty acids, the esters of such acids, and alcohols, alone or in combination.

A compound which may be suitable for use as the organic phase is any compound belonging to the categories mentioned which is in liquid form at ambient temperature (20-30° C.) and/or at the temperature at which the emulsion is prepared.

As organic waxes/fats/oils of animal origin mention may be made, inter alia, of cachalot oil, whale oil, seal oil, shark oil, cod liver oil, lard and mutton fat (tallows), perhydrosqualene and beeswax, alone or in a mixture.

By way of examples of organic waxes/fats/oils of plant origin, mention may be made, inter alia, of rapeseed oil, sunflower oil, peanut oil, olive oil, walnut oil, corn oil, soybean oil, avocado oil, linseed oil, hemp oil, grapeseed oil, copra oil, palm oil, cottonseed oil, babassu oil, jojoba oil, sesame oil, castor oil, macadamia oil, sweet almond oil, carnauba wax, karite butter, cocoa butter, and peanut butter, alone or in a mixture.

As regards the mineral waxes/oils, mention may be made, inter alia, of naphthenic oils, liquid paraffins (vaseline), isoparaffins, and paraffin waxes, alone or in a mixture.

The products obtained from the alcoholysis of the aforementioned oils may also be used.

Among essential oils mention may be made, without any intention of limitation thereto, of oils and/or essences of mint, spearmint, peppermint, menthol, vanilla, cinnamon, bay, aniseed, eucalyptus, thyme, sage, cedar leaf, nutmeg, citrus (lemon, lime, grapefruit, orange), and fruits (apple, pear, peach, cherry, prune, strawberry, raspberry, apricot, pineapple, grape, etc.), alone or in mixtures.

As regards the fatty acids, these acids are more particularly saturated or unsaturated mono- or polycarboxylic acids containing 10 to 40 carbon atoms, more particularly 18 to 40 carbon atoms, and may comprise one or more conjugated or nonconjugated ethylenic unsaturations. It should be noted that said acids may optionally comprise one or more hydroxyl groups.

As examples of saturated fatty acids mention may be made of palmitic, stearic and behenic acids.

As examples of unsaturated fatty acids mention may be made of myristoleic, palmitoleic, oleic, erucic, linoleic, linolenic, arachidonic, and ricinoleic acids and also mixtures thereof.

As regards the fatty acid esters, mention may be made, for example, of the esters of the above-listed acids for which the part deriving from the alcohol contains 1 to 6 carbon atoms, such as methyl, ethyl, propyl and isopropyl esters, etc.

As examples of alcohols mention may be made of those which contain 2 to 40 carbon atoms, are saturated or unsaturated, and may optionally comprise one or more additional hydroxyl groups. More particularly, ethanol and the alcohols corresponding to the aforementioned acids may be suitable.

The organic phase may also be selected from mono-, di- and tri-glycerides.

It will be appreciated that the organic phase may comprise a mixture of two or more of the compounds just listed.

Finally, it is specified that the invention does not exclude the disperse organic phase comprising an amount of water not exceeding the solubility limit of water in said phase (at a temperature of between 20 and 30° C.).

As for the continuous phase of the emulsion, it is specified that the silicone is selected such that it is immiscible with the disperse organic phase.

By immiscible is meant any liquid which, at ambient temperature, has a solubility in another liquid that does not exceed 10% by weight.

Among the silicones which may be suitable for the implementation of the invention, mention may be made of, alone or in mixtures, silicones composed in whole or in part of units of formula:

R′₃₋ₐRₐSiO_{1/2} (unit M) and/or R₂SiO (unit D) in which formulae:

a is an integer from 0 to 3;

the radicals R are identical or different and represent:

a saturated or unsaturated aliphatic hydrocarbon group containing 1 to 10 carbon atoms;

an aromatic hydrocarbon group containing 6 to 13 carbon atoms;

a polar organic group bonded to the silicon by an Si—C or Si—O—C bond;

the radicals R′ are identical or different and represent:

a saturated or unsaturated aliphatic hydrocarbon group containing 1 to 10 carbon atoms;

an aromatic hydrocarbon group containing 6 to 13 carbon atoms;

an —OH function;

an amino- or amido-functional group containing 1 to 6 carbon atoms, bonded to the silicon by an Si—N bond.

Preferably at least 80% of the radicals R represent a methyl group.

These silicones may optionally comprise, preferably less than 5 mol % of, units of formulae T and/or Q:

RSiO_{3/2} (unit T) and/or SiO₂ (unit Q) in which formula R is as defined above.

By way of examples of aliphatic or aromatic hydrocarbon radicals R mention may be made of the following groups:

alkyl, preferably C₁-C₁₀ alkyl, optionally halogenated, such as methyl, ethyl, octyl or trifluoropropyl;

alkoxyalkylene, more particularly C₂-C₁₀, preferably C₂-C₆, such as —CH₂—CH₂—O—CH₃;

alkenyl, preferably C₂-C₁₀ alkenyl, such as vinyl, allyl, hexenyl, decenyl or decadienyl;

alkenyloxyalkylene such as —(CH₂)₃—O—CH₂—CH₂—O—CH═CH₂, or alkenyloxyalkoxyalkyl such as —(CH₂)₃—OCH₂—CH₂—O—CH═CH₂, in which the alkyl moieties are preferably C₁-C₁₀ and the alkenyl moieties are preferably C₂-C₁₀;

aryls, preferably C₆-C₁₃, such as phenyl.

By way of examples of polar organic groups R mention may be made of the following groups:

hydroxy-functional groups such as alkyl groups substituted by one or more hydroxyl or di(hydroxyalkyl)amino groups and optionally interrupted by one or more divalent hydroxyalkylamino groups. By alkyl is meant a hydrocarbon chain, preferably C₁-C₁₀, more preferably C₁-C₆; examples of these groups are —(CH₂)₃—OH;

—(CH₂)₄N(CH₂CH₂OH)₂; —(CH₂)₃—N(CH₂CH₂OH)—CH₂;

—CH₂—N(CH₂CH₂OH)₂;

amino-functional groups such as alkyl substituted by one or more amino or aminoalkylamino groups, where alkyl is as defined above; examples thereof are —(CH₂)₃—NH₂; (CH₂)₃—NH—(CH₂)₂NH₂;

amido-functional groups such as alkyl substituted by one or more acylamino groups and optionally interrupted by one or more divalent alkyl-CO—N< groups, where alkyl is as defined above and acyl represents alkylcarbonyl, where one example is the group —(CH₂)₃—N(COCH₃)— (CH₂)₂NH(COCH₃);

carboxy-functional groups such as carboxyalkyl optionally interrupted by one or more oxygen or sulfur atoms, in which alkyl is as defined above; one example is the group —CH₂—CH₂—S—CH₂—COOH.

By way of examples of radicals R′ mention may be made of the following groups:

alkyl, preferably C₁-C₁₀ alkyl, optionally halogenated, such as methyl, ethyl, octyl or trifluoropropyl;

aryl, preferably C₆-C₁₃, such as phenyl;

amino-functional groups such as alkyl or aryl substituted by amino, alkyl being preferably C₁-C₆ and aryl denoting a cyclic aromatic hydrocarbon group, preferably C₆-C₁₃, such as phenyl; examples thereof are ethylamino and phenylamino;

amido-functional groups such as alkylcarbonylamino, where alkyl is preferably C₁-C₆; examples thereof are methylacetamido.

By way of specific examples of “units D” mention may be made of: (CH₃)₂SiO; CH₃(CH═CH₂)SiO; CH₃(C₆H₅)SiO; (C₆H₅)₂SiO; CH₃(CH₂—CH₂—CH₂OH)SiO.

By way of specific examples of “units M” mention may be made of: (CH₃)₃SiO_{1/2}; (CH₃)₂(OH)SiO_{1/2}; (CH₃)₂(CH═CH₂)SiO_{1/2}; (OCH₃)₃SiO_{1/2}; [O—C(CH₃)═CH₂]₃SiO_{1/2}; [ON═C(CH₃)]₃SiO_{1/2}; (NH—CH₃)₃SiO_{1/2}; (NH—CO—CH₃)₃SiO_{1/2}.

By way of specific examples of “units T” mention may be made of: CH₃SiO_{3/2}; (CH═CH₂)SiO_{3/2}.

When the silicones contain reactive and/or polar radicals R (such as OH, vinyl, allyl, hexenyl, aminoalkyls, etc.), these radicals do not generally represent more than 5% of the weight of the silicone, and preferably not more than 1% of the weight of the silicone.

As continuous phase of the emulsion, it is possible to use volatile oils such as hexamethyldisiloxane, octamethyldisiloxane, decamethyltetrasiloxane, dodecamethylpentasiloxane, tetradecamethylhexasiloxane, hexadecamethylhexasiloxane; heptamethyl-3[(trimethylsilyl)oxy]trisiloxane, hexamethyl-3,3 bis[(trimethylsilyl)oxy]trisiloxane; hexamethylcyclotrisiloxane, octamethylcyclotetrasiloxane, decamethylcyclopentasiloxane, dodecamethylcyclohexasiloxane, pentamethyl-[(trimethylsilyl)oxy]cyclotrisiloxane.

It is likewise possible to employ nonvolatile silicones such as polydimethylsiloxane and α,ω-bis(hydroxy)polydimethylsiloxane gums and oils and also α,ω-bis(hydroxy)polydimethylsiloxane, polyphenylmethylsiloxane and polydimethylsiloxane gums may likewise be used as continuous phase of the emulsion.

Preference is given more particularly to α,ω-bis(trimethyl)polydimethylsiloxane oils and α,ω-bis-(hydroxy)polydimethylsiloxane oils.

As representative silicones which are particularly suitable for the present invention mention may be made in particular of the silicones of polydimethylsiloxane (dimethicone) and diphenyldimethicone type.

The proportion by weight of disperse organic phase relative to silicone continuous phase is more particularly between 10/90 and 90/10, preferably between 30/70 and 50/50.

One of the features of the invention is that the oil-in-oil emulsion is stabilized by the presence of at least one comb or block copolymer one fraction of which is soluble in the disperse phase and the other in the continuous phase. Moreover, Bancroft's rule may be applied to this polymer, since the fraction soluble in the continuous phase is greater than the fraction soluble in the disperse phase.

More precisely, said copolymer is selected from those which are soluble in the silicone. More particularly, soluble copolymers are those which, when mixed with the silicone, at a concentration of between 0.1 and 10% by weight of said silicone and at 20° C., are in the form of a solution within all or part of the concentration range indicated.

Moreover, said copolymer is selected from those which, when mixed with the disperse organic phase, at a concentration of between 0.1 and 10% by weight of said organic phase and at 20° C., are in the form of a dispersion within all or part of the concentration range indicated. In effect, the copolymer is only partly soluble in the organic phase.

Finally, said copolymer employed is one which, at a given concentration within the range indicated above and at 20° C., is present simultaneously in the form of a solution in the silicone and in the form of a dispersion in the organic phase.

Simply by way of indication it is specified that the copolymer has a number-average molar mass of less than or equal to 100000 g/mol, more particularly between 1000 and 50000 g/mol. The number-average molar mass is given here with an absolute value, and may be advantageously determined by combining analysis by Malls (multiangle light scattering) with analysis by gel permeation chromatography. It should be specified that this method is particularly appropriate for copolymers of any mass. For copolymers of low mass, especially masses less than or equal to around 20000 g/mol, it is also possible to use NMR, which also gives an absolute value for copolymer masses.

Advantageously, if each of the blocks of the copolymer represents one polymer (same size and composition as the blocks), then the monomers constituting each of the blocks will be selected such that each polymer is soluble, under the above-mentioned temperature and concentration conditions, either in the silicone (for the polymer deriving from the blocks soluble in the silicone) or in the organic phase (for the polymer deriving from the blocks soluble in the organic phase).

More particularly, the fraction of the copolymer which is soluble in the silicone derives from a polysiloxane, which may be selected from the silicones mentioned above.

The polysiloxane preferably carries reactive functional groups, such as the functions —OH and —NH₂, among others.

As regards the fraction soluble in the organic phase, this fraction derives, preferably, from the polymerization of at least one monomer selected from the following monomers:

esters of linear, branched, cyclic or aromatic monocarboxylic or polycarboxylic acids comprising at least one ethylenic unsaturation;

α,β-ethylenically unsaturated nitrites, vinyl ethers, vinyl esters, vinylaromatic monomers, vinyl halides or vinylidene halides;

linear or branched, aromatic or nonaromatic hydrocarbon monomers comprising at least one ethylenic unsaturation;

alone or in mixtures, and also the macromonomers deriving from such monomers.

More specifically said monomers may be selected from:

the esters of (meth)acrylic acid with an alcohol containing 1 to 12 carbon atoms, such as methyl (meth)acrylate, ethyl (meth)acrylate, propyl (meth)acrylate, n-butyl (meth)acrylate, t-butyl (meth)acrylate, isobutyl (meth)acrylate, 2-ethylhexyl acrylate and hydroxyethyl (meth)acrylate;

vinyl acetate (which makes it possible to obtain partly or totally deacetylated polyvinyl alcohol), vinyl Versatate®, vinyl propionate, vinyl chloride, vinylidene chloride, methyl vinyl ether, ethyl vinyl ether, (meth)acrylonitrile, N-vinylpyrrolidone, vinylformamide, vinylacetamide and vinylamine,

(meth)acrylamide, N-alkyl (meth)acrylamide such as isopropyl acrylamide, and N-methylol (meth)acrylamide;

styrene, α-methylstyrene, vinyltoluene, butadiene, chloroprene and isoprene;

alone or in mixtures, and the macromonomers deriving from such monomers.

It will be recalled that the term “macromonomers” denotes a macromolecule carrying one or more polymerizable functions.

It should be noted that the fraction soluble in the organic phase of the copolymer employed as stabilizer of the emulsion may be obtained from aforementioned monomers in combination with monomers of different chemical nature, such as, for example, ionic or nonionic hydrophilic monomers.

By way of example of ionic hydrophilic monomers, more particularly anionic or potentially anionic depending on the pH conditions, mention may be made in particular of:

linear, branched, cyclic or aromatic monocarboxylic or polycarboxylic acids, the N-substituted derivatives of such acids, and monoesters of polycarboxylic acids, comprising at least one ethylenic unsaturation;

linear, branched, cyclic or aromatic vinylcarboxylic acids;

amino acids comprising one or more ethylenic unsaturations;

alone or in mixtures, their sulfonic or phosphonic derivatives, the macromonomers deriving from such monomers, and the corresponding salts.

Possible examples of suitable monomers of this type, in acid form or in the alkali metal (sodium, potassium) or ammonium salt form, include the following:

(meth)acrylic acid, 2-propene-1-sulfonic acid, methallylsulfonic acid, styrenesulfonic acid, α-acrylamidopropanesulfonic acid, 2-sulfoethylene methacrylate, sulfopropyl(meth)acrylic acid, bis-sulfopropyl(meth)acrylic acid and the phosphate monoester of hydroxyethylmethacrylic acid;

vinylsulfonic acid, vinylbenzenesulfonic acid, vinylphosphonic acid and vinylbenzoic acid;

N-methacryloylalanine and N-acryloylhydroxyglycine;

alone or in mixtures, and the macromonomers deriving from such monomers.

Nonionic hydrophilic monomers include the amides of linear, branched, cyclic or aromatic monocarboxylic or polycarboxylic acids containing at least one ethylenic unsaturation or derivatives such as (meth)acrylamide and N-methylol(meth)acrylamide; certain esters deriving from (meth)acrylic acid, such as, for example, 2-ethylhydroxyl (meth)acrylate; and vinyl esters which make it possible to obtain polyvinyl alcohol blocks after hydrolysis, such as vinyl acetate, vinyl Versatate®, and vinyl propionate.

If the copolymer forming part of the composition of the emulsion is obtained in part from hydrophilic monomers, it is specified that their amount is such that the copolymer meets the criteria set out previously, namely the fact that one fraction of said copolymer is soluble in the continuous phase of the emulsion, the other in the disperse phase, the fraction soluble in the liquid constituting the continuous phase of the emulsion being higher than that soluble in the disperse phase.

According to one preferred embodiment of the present invention the polymer forming the part of the composition of the emulsion is a linear block polymer. Said copolymer preferably comprises at least three blocks.

The copolymers which can be used in the context of the present invention may be obtained advantageously by a free-radical route, preferably controlled.

With preference it is possible to obtain such polymers by employing a process for preparing, by thermal activation, hybrid silicone and organic copolymers comprising units (I):

RₓU_{y}SiO_{[4-(x+y)]/2} (I)

in which:

x is 0, 1, 2 or 3, y is 0, 1, 2 or 3, where 2≦(x+y)<≦3 and y is other than 0 for at least one of the units of the hybrid copolymer,

the symbols R, which are identical or different, represent:

a linear or branched alkyl radical containing 1 to 8 carbon atoms, optionally substituted by at least one halogen, preferably fluorine, the alkyl radicals being preferably methyl, ethyl, propyl, octyl and 3,3,3-trifluoropropyl,

a cycloalkyl radical containing between 5 and 8 cyclical carbon atoms, which is optionally substituted,

an aryl radical containing between 6 and 12 carbon atoms, which may be substituted, preferably phenyl or dichlorophenyl,

an aralkyl moiety having an alkyl moiety containing between 5 and 14 carbon atoms and an aryl moiety containing between 6 and 12 carbon atoms, optionally substituted on the aryl moiety by halogens, alkyls and/or alkoxys containing 1 to 3 carbon atoms,

the symbols U which are identical or different, represent (II):

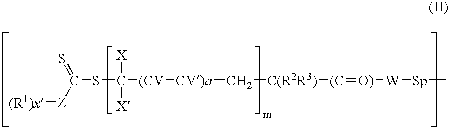

in which:

x′=1, 2, 3 or 4, depending on the valence of Z,

Z, which are identical or different, represent a carbon, sulfur, oxygen, phosphorus or nitrogen atom and/or a free valence,

R¹, which are identical or different, represent:

an optionally substituted alkyl, acyl, aryl, alkene or alkyne group (i),

a saturated or unsaturated carbon ring (ii), optionally substituted and/or aromatic, and/or

a saturated or unsaturated heterocycle (iii), optionally substituted,

it being possible for these radicals (i), (ii) and (iii) to be substituted advantageously by: substituted phenyl groups, substituted aromatic groups, or the following groups: alkoxycarbonyl, aryloxycarbonyl (—COOR⁵), carboxyl (—COOH), acyloxy (—O₂CR⁵), carbamoyl (—CONR⁵)₂, cyano (—CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups having a hydrophilic or ionic character such as the alkali metal salts of carboxylic acids, the alkali metal salts of sulfonic acid, polyalkylene oxide chains (PEO, PPO), cationic substituents (quaternary ammonium salts), with R⁵, which are identical or different, representing an alkyl or aryl group, and/or a polymer chain,

a group (iv) of formula —CₙF₍₂ₙ₊₁₎ where n is between 1 and 20,

a group (v) of the following formula:

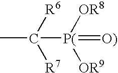

in which:

R⁶ and R⁷, which are identical or not, are selected from a halogen group, —NO₂, —SO₃R¹⁰, —NCO, —CN, —OR¹⁰, —SR^{10,} N(R¹⁰)₂, —COOR¹⁰, —O₂CR¹⁰, —CON(R¹⁰)₂, —NCO(R¹⁰)₂ and —CₙF₂ₙ₊₁₎, where n is between 1 and 20, and is preferably 1;

where R¹⁰ represents:

a hydrogen atom,

or an alkyl, alkenyl, alkynyl, cycloalkenyl, cycloalkynyl, alkaryl, aralkyl, heteroaryl or aryl radical optionally fused with an aromatic or nonaromatic heterocycle, it being possible for these radicals to be optionally substituted by:

one or more identical or different groups selected from halogen atoms, ═O, ═S, —OH, alkoxy, SH, thioalkoxy, NH₂, mono- or dialkylamino, CN, COOH, ester, amide, and CₙF₍₂ₙ₊₁) and/or optionally interrupted by one or more atoms selected from O, S, N and P;

or a heterocyclic group optionally substituted by one or more groups as defined above;

or R⁶ and R⁷, together with the carbon atom to which they are attached, form a group ═O, ═S, a hydrocarbon ring or a heterocycle;

and R⁸ and R⁹, which are identical or different, represent a group as defined above for R¹⁰, or together form a C₂-C₄ hydrocarbon chain, optionally interrupted by a heteroatom selected from O, S and N;

V and V′, which are identical or different, represent H, an alkyl group or a halogen,

X and X′, which are identical or different, represent H, a halogen or a group R⁴, OR⁴, O₂COR⁴, NHCOH, OH, NH₂, NHR⁴, N(R⁴)₂, (R⁴)₂N⁺O⁻, NHCOR⁴, CO₂H, CO₂R⁴, CN, CONH₂, CONHR⁴ or CONR⁴₂, in which R⁴ is selected from alkyl, aryl, aralkyl, alkaryl alkene or organosilyl groups, which are optionally perfluorinated and optionally substituted by one or more carboxyl, epoxy, hydroxyl, alkoxy, amino, halogen or sulfonic groups,

R² and R³, which are identical or different, represent:

an optionally substituted alkyl, acyl, aryl, alkene or alkyne group (i),

a saturated or unsaturated carbon ring (ii), optionally substituted and/or aromatic,

a saturated or unsaturated heterocycle (iii), optionally substituted,

a hydrogen atom or the following groups: alkoxycarbonyl, aryloxycarbonyl (—COOR⁵) carboxyl (—COOH), acyloxy (−O₂CR⁵), carbamoyl (—CONR⁵₂), cyano (—CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups having a hydrophilic or ionic character such as the alkali metal salts of carboxylic acids, the alkali metal salts of sulfonic acid, polyalkylene oxide chains (PEO, PPO), and cationic substituents (quaternary ammonium salts), with R⁵, which are identical or different, representing an alkyl or aryl group and/or a polymer chain,

it being possible for the radicals (i), (ii) and (iii) to be substituted advantageously by: substituted phenyl groups, substituted aromatic groups, or the following groups: alkoxycarbonyl, aryloxycarbonyl (—COOR⁵), carboxyl (—COOH), acyloxy (−O₂CR⁵), carbamoyl (—CONR⁵₂), cyano (—CN), alkylcarbonyl, alkylarylcarbonyl, arylcarbonyl, arylalkylcarbonyl, phthalimido, maleimido, succinimido, amidino, guanidimo, hydroxyl (—OH), amino (—NR⁵₂), halogen, allyl, epoxy, alkoxy (—OR⁵), S-alkyl, S-aryl, groups having an ionic or hydrophilic character such as the alkali metal salts of carboxylic acids, the alkali metal salts of sulfonic acid, polyalkylene oxide chains (PEO, PPO), cationic substituents (quaternary ammonium salts), with R⁵, which are identical or different, representing an alkyl or aryl group and/or a polymer chain,

W, which are identical or different, represent a divalent radical selected from —O—, —NR⁴—, —NH—, and —S—,

Sp, which are identical or different, represent a coupling unit composed of a divalent organic radical of —(CH₂)_{x′″}—, in which x′″ is between 1 and 20, it being possible for this radical to be substituted and/or to contain at least one heteroatom,

a=0 or 1,

m≧1, and, when m>1, the repeating units of index m are identical or different,

in which process the following are contacted with one another:

at least one ethylenically unsaturated monomer of the formula (III):

CXX′(═CV—CV′)ₐ═CH₂

a precursor silicone compound comprising identical or different units of formula (IV):

RₓU′_{y}SiO_{[4-(x+y)]/2}

in which:

R, x and y correspond to the values given above,

and the monovalent radical U′ is in accordance with the following formula (V):

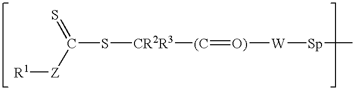

and a free-radical polymerization initiator.

The free-radical polymerization initiator may be selected from initiators conventionally used in free-radical polymerization. It may be, for example, one of the following initiators:

hydrogen peroxides, such as tert-butyl hydroperoxide, cumene hydroperoxide, t-butyl peroxyacetate, t-butyl peroxybenzoate, t-butyl peroxyoctoate, t-butyl peroxyneodecanoate, t-butyl peroxyisobutarate, lauroyl peroxide, t-amyl peroxypivalate, t-butyl peroxypivalate, dicumyl peroxide, benzoyl peroxide, potassium persulfate, and ammonium persulfate,

azo compounds such as: 2,2′-azobis(isobutyronitrile), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]propionamide, 2,2′-azobis(2-methyl-N-hydroxyethyl]propionamide, 2,2′-azobis(N,N′-dimethyleneisobutyramidine) dichloride, 2,2′-azobis(2-amidinopropane) dichloride, 2,2′-azobis(N,N′-dimethyleneisobutyramide), 2,2′-azobis(2-methyl-N-[1,1-bis(hydroxymethyl)-2-hydroxyethyl]propionamide, 2,2′-azobis(2-methyl-N-[1,1-bis(hydroxymethyl)ethyl]propionamide), 2,2′-azobis[2-methyl-N-ethyl]propionamide, 2,2′-azobis(2-methyl-N-(2-hydroxyethyl)propionamide] and 2,2′-azobis(isobutyramide) dihydrate,

redox systems comprising combinations such as: mixtures of hydrogen peroxide, alkyl peroxide, peresters, percarbonates and the like and arbitrary iron salts, titanium salts, zinc formaldehyde sulfoxylate or sodium formaldehyde sulfoxylate, and reducing sugars; ammonium or alkali metal persulfates, perborate or perchlorate in combination with an alkali metal bisulfite, such as sodium metabisulfite, and reducing sugars; alkali metal persulfates in combination with an arylphosphinic acid, such as benzenephosphonic acid and other, similar compounds, and reducing sugars.

The amount of initiator to be used is determined such that the amount of radicals generated is not more than 20 mol % relative to the amount of silicone precursor compound (IV), preferably not more than 5 mol %.

As ethylenically unsaturated monomer use is made of those mentioned above for defining the fraction of the copolymer soluble in the disperse organic phase of the emulsion.

It should be specified, moreover, that butadiene and chloroprene correspond to the case in which a=1 in formula (I) and (III),

For the preparation of the hybrid copolymers of formula (I) for which X═H and X′=NH₂ it is preferred to use, as ethylenically unsaturated monomers, vinylamine amides: for example, vinylformamide or vinylacetamide. The copolymer obtained is then hydrolyzed at acidic or basic pH.

For the preparation of the hybrid copolymers of formula (I) for which X═H and X′═OH, it is preferred to use as ethylenically unsaturated monomers, the vinyl esters of carboxylic acid, such as, for example, vinyl acetate. The copolymer obtained is then hydrolyzed at an acid or basic pH.

The types and amounts of copolymerizable monomers employed according to the present invention vary depending on the particular end application for which the hybrid copolymer is intended.

According to a first preferred version, the hybrid silicone and organic copolymer is composed of a linear silicone skeleton comprising from 1 to 300 units of formula (I), preferably from 1 to 200, and carrying from 1 to 50 radicals U, preferably from 1 to 10.

According to a second version, at least one of the monovalent radicals U′ is preferably of formula (VI):

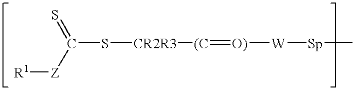

in which R² and R³, which are identical or different, represent a hydrogen atom or an optionally substituted alkyne, alkene, aryl, acyl, alkyl, alkoxycarbonyl or cyano group; and W has a definition identical to that given above, and is preferably an oxygen atom. Examples that will be mentioned include the silicone precursors in which U′ corresponds to:

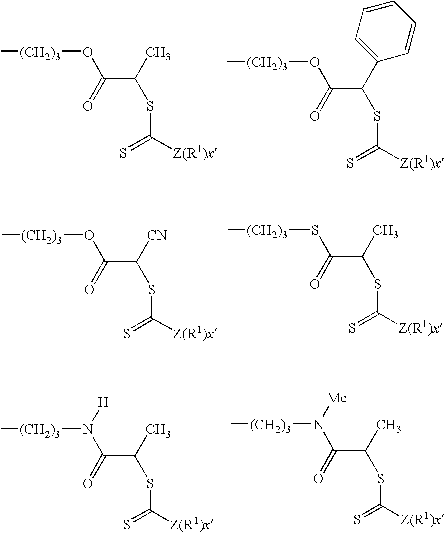

According to a third version of the invention, at least some of the monovalent radicals U′ of the silicone precursor(s) (IV) and thus at least some of the groups U of the hybrid copolymer obtained are such that Z is an oxygen atom and/or a sulfur atom.

According to a fourth variant, in addition to the use of formula (I), the hybrid silicone and organic copolymer according to the invention may comprise units RₓU_{y}F_{z}SiO_{[4-(x+y+z)]/2} (XIV) in which:

x is 0, 1, 2 or 3, y is 0, 1, 2 or 3, and z is 1, 2 or 3, where 2≦(x+y+z)≦3,

and F is a group carrying at least one functional group such as hydroxyl, alkoxy, thiol, amine, epoxy and/or polyether.

These groups F may optionally provide complementary and/or additional properties to the hybrid copolymers prepared according to the process of the invention. They may in particular be present initially within the silicone precursor of formula (IV).

In addition to the hybrid copolymers with homopolymeric organic segments, the process which has just been described makes it possible to prepare hybrid polymers carrying organic groups in blocks (i.e. multiblock polymers). To do this, the process consists in repeating the implementation of the preparation process described above, using:

monomers that are different than in the previous implementation, and

in place of the precursor silicone compound (II), the hybrid copolymer comprising block units (I) obtained from the previous implementation.

According to this process for preparing block copolymers, when it is desired to obtain copolymers having homogeneous blocks without a composition gradient, and if all the successive polymerizations are conducted in the same reactor, it is essential that all of the monomers used in one step should have been consumed before the polymerization of the following step beings, and thus before the new monomers are introduced.

As for the process for polymerizing a monoblock copolymer, this process for polymerizing block copolymers-has the advantage of leading to block copolymers which have a low polydispersity index. It also allows the molecular mass of the block polymers to be controlled.

The precursor silicone compound of general formula (IV) used in the process for preparing hybrid copolymers according to the invention may be obtained by reacting:

(i) a silicone comprising units of formula (VII):

RₓU″_{y}SiO_{[4-(x+y)]/2}

in which the monovalent radical U″ is in accordance with the following formula (VIII):

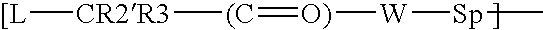

in which:

W and Sp have definitions identical with those given above,

L is a nucleophilic group, for example: Br⁻, Cl⁻, I⁻, OTs⁻, OMs⁻, (C₆H₆)—(C═O)—O⁻, (CH₃)—(C═O)—O⁻, (CF₃—(C═O)—O⁻,

(ii) with a compound selected from those of following general formulae (IX), (X) or (XI):

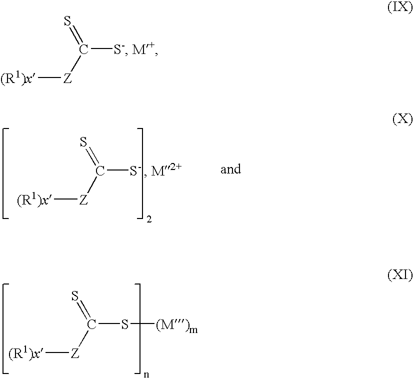

in which:

M′⁺ represents K⁺, Na⁺, NR₄⁺ or PR₄⁺, the definition of R being similar to that given for R in formula (I),

M″²⁺ represents an alkaline earth metal such as Ca²⁺, Ba²⁺ and Sr²⁺⁺,

M′″ represents Zn, Cd, m is 1 or 2, n is 1, 2, 3 or 4, and preferably m is 1 and n is 2.

This silicone of formula (VII) can be obtained in particular from (i) a silicone comprising units of formula (XII): RₓU′″_{y}SiO_{[4-(x+y)]/}2, in which the monovalent radical U′″ is of formula (XIII): —Sp—WH, and (ii) a compound of formula:

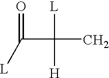

The polymerization may be conducted without solvent, in solution or in emulsion. It is preferably implemented in emulsion.

The process is preferably implemented in semicontinuous fashion.

The temperature may vary between the ambient temperature and 150° C., depending on the nature of the monomers used.

In general, during the polymerization, the instantaneous copolymer content relative to the instantaneous amount of monomer and copolymer is between 50 and 99% by weight, preferably between 75 and 99%, more preferably still between 90 and 99%. This content is maintained, in a known manner, by controlling the temperature and the rate of addition of the reactants and of the polymerization initiator.

Finally, the process is generally implemented in the absence of a UV source.

It should be noted that it may be advantageous to modify chemically the xanthate ends of the resultant copolymer, employing any method known to the skilled worker, such as, for example, a step of hydrolysis.

The process and the polymers obtained by this process are described in French application no. 00/09722, filed Jul. 25, 2000.

Other methods of synthesizing this type of copolymer may be employed, particularly that described in international applications WO 00/71606 and WO 00/71607.

According to one particular embodiment of the invention the amount of copolymer represents from 0.5 to 10% by weight of the disperse phase, preferably between 1 and 4% by weight relative to the same reference.

The disperse phase of the emulsion may further comprise at least one active substance soluble in said phase (hydrophobic active substance).

More particularly the active substance or substances are selected from active substances which can be used in the field of the treatment of the skin and/or hair. Included among these in particular, alone or in mixtures, are the following:

lipophilic vitamins, such as vitamin A and its derivatives, especially its esters, such as the acetate, palmitate, and propionate, vitamin B2, panthothenic acid, vitamin D, and vitamin E;

UV absorbers, such as the aminobenzoate derivatives of PABA and PARA type, salicylates, cinnamates, anthranilates, dibenzoylmethanes, and camphor derivatives; antiaging agents such as, in particular, retinoids, fat-soluble vitamins, vitamin C derivatives such as the esters of the acetate, propionate, and palmitate type; ceramides, pseudoceramides, phospholipids, fatty acids, fatty alcohols, cholesterol, sterols, and mixtures thereof. As preferred fatty acids and preferred alcohols mention may be made more particularly of those which possess linear or branched alkyl chains containing 12 to 20 carbon atoms. The compound involved may in particular be linoleic acid;

anticellulite agents, such as, in particular, isobutylmethylxanthine and theophylline;

antiacne agents, such as, for example, resorcinol, resorcinol acetate, benzoyl peroxide, and numerous natural compounds; bactericides;

antimicrobial agents may be selected from thymol, menthol, triclosan, 4-hexylresorcinol, phenol, eucalyptol, benzoic acid, benzoic peroxide, and butyl paraben;

aromas, perfumes, and essential oils, such as, in particular, the essential oils/essences cited above in the context of the description of L2, but also ambergris, benjamin, clove, civet, jasmine, sandalwood, vetiver, musk, myrrh, and iris, or else aldehydes and esters, such as, for example, cinnamyl acetate, cinnamaldehyde, p-methylanisole, acetaldehyde, benzaldehyde, vanillin, decanal, nerol, citral, 2,6-dimethyloctanal, and 2-ethylbutyraldehyde;

fixing resins, particularly selected from those which will be described later on below, and also fixatives and/or styling agents for the hair, such as, for example, the vinyl acetate/crotonate/vinyl neodecanoate copolymers sold under the name Resyn® 28-2942 and Resyn® 28-2930 from National Starch.

Where the disperse phase comprises one or more hydrophobic active substances, and where the active substance or substances are not used additionally as disperse organic phase, their amount represents more particularly 10 to 50% by weight of said disperse organic phase.

It should be noted that the disperse organic phase itself may be considered as a hydrophobic active substance. Conversely, the active substance or substances may be considered as a disperse phase.

The invention additionally provides a dispersion of the emulsion comprising liquids L1 and L2, dispersed in an external aqueous phase (continuous external aqueous phase).

According to this subject of the invention, the external aqueous phase comprises at least one nonionic surfactant and/or at least one nonionic amphiphilic polymer, alone or in mixtures, optionally in combination with one or more anionic surfactants and/or one or more anionic amphiphilic polymers.

The surfactant is preferably selected from nonionic polyoxyalkylenated surfactants which are at least partly miscible in the aqueous phase.

The polyoxyalkylenated surfactant of the external aqueous phase is selected advantageously from the following surfactants, alone or in a mixture:

alkoxylated fatty alcohols; alkoxylated triglycerides;

alkoxylated fatty acids; alkoxylated sorbitan esters, alkoxylated fatty amines; alkoxylated di(1-phenylethyl)phenols; alkoxylated tri(1-phenylethyl)phenols; alkoxylated alkylphenols; the number of alkoxylated units, more particularly oxyethylenated and/or oxypropylenated units, is such that the HLB value is greater than or equal to 10.

As regards the anionic or nonionic amphiphilic polymers, a polymer is employed which comprises at least two blocks, one of them being hydrophilic and the other hydrophobic. That which was indicated previously in the context of the description of the monomers which are hydrophilic and nonionic, ionic and those which are hydrophobic and can be used for the preparation of the comb or block copolymers.

Said amphiphilic polymers may advantageously be obtained by what is termed living or controlled free-radical polymerization. For nonlimitative examples of controlled or living polymerization processes reference may be made in particular to the applications WO 98/58974, WO 00/75207 and WO 01/42312 (xanthate), WO 98/01478 (dithio esters), WO 99/03894 (nitroxides); WO 99/31144 (dithiocarbamates), WO 2/26836 (dithiocarbazates); WO 02/10223 (dithiophosphoroesters), WO 96/30421 (atom transfer polymerization—ATRP).

The amphiphilic polymers may also be obtained by anionic polymerization.

They may likewise be prepared by employing ring-opening polymerizations (especially anionic), or by chemical modification of the polymer.

More particularly, as regards the present amphiphilic polymer, preferably polyoxyalkylenated, of the external aqueous phase, it may be selected from polymers which are at least partly miscible in the external aqueous phase and preferably from polyethylene glycol/polypropylene glycol/polyethylene glycol triblock copolymers.

It is specified that polymers of polyvinyl alcohol type or of polyacrylic acid/polybutyl acrylate/polyacrylic acid triblock type may be used for this purpose.

The amount of anionic surfactant and/or of nonionic amphiphilic polymer is advantageously between 0.5 and 10% by weight relative to the weight of the emulsion (L2 in L1), preferably between 1 and 5% by weight of the emulsion.

Among the anionic surfactants which can be combined with at least one nonionic surfactant and/or at least one nonionic polymer, mention may be made, inter alia, of the following, alone or in mixtures:

alkyl ester sulfonates, for example of formula R—CH(SO₃M)—COOR′, in which R represents a C₈-C₂₀, preferably C₁₀-C₁₆, alkyl radical, R′ a C₁-C₆, preferably C₁-C₃, alkyl radical, and M an alkali metal cation (sodium, potassium or lithium), a substituted or unsubstituted ammonium cation (methyl-, dimethyl-, trimethyl-, tetramethylammonium, dimethylpiperidinium, etc.) or a cation derived from an alkanolamine (monoethanolamine, diethanolamine, triethanolamine, etc.). Very particular mention may be made of the methyl ester sulfonates whose radical R is C₁₄-C₁₆; alkylbenzenesulfonates, more particularly C₉-C₂₀ alkylbenzenesulfonates, primary or secondary alkylsulfonates, especially C₈-C₂₂ alkylsulfonates, alkylglycerol sulfonates, sulfonated polycarboxylic acids, such as, for example, those described in GB 1082179, and paraffinsulfonates;

alkyl sulfates for example of formula ROSO₃M, in which R represents a C₁₀-C₂₄, preferably C₁₂-C₂₀, alkyl or hydroxyalkyl radical, M representing a hydrogen atom or a cation of the same definition as above, and their polyalkoxylated (ethoxylated (EO), propoxylated (PO), or combinations thereof) derivatives, such as, for example, sodium dodecyl sulfate;

alkyl ether sulfates for example of formula RO(CH₂CH₂O)ₙSO₃M, in which R represents a C₁₀-C₂₄, preferably C₁₂-C₂₀, alkyl or hydroxyalkyl radical, M representing a hydrogen atom or a cation of the same definition as above, n bearing generally from 1 to 4, and their polyalkoxylated (ethoxylated (EO), propoxylated (PO), or combinations thereof) derivatives, such as, for example, lauryl ether sulfate with n=2;

alkylamide sulfates, for example of formula RCONHR′OSO₃M, in which R represents a C₂-C₂₂, preferably C₆-C₂₀, alkyl radical, R′ a C₂-C₃ alkyl radical, M representing a hydrogen atom or a cation of same definition as above, and their polyalkoxylated (ethoxylated (EO), propoxylated (PO), or combinations thereof) derivatives;

salts of saturated or unsaturated fatty acids, for example such as the C₈-C₂₄, preferably C₁₄-C₂₀, compounds, N-acyl-N-alkyltaurates, alkylisethionates, alkylsuccinamates and alkylsulfosuccinates, sulfosuccinate monoesters or diesters, N-acylsarcosinates, and polyethoxycarboxylates; and

alkyl and/or alkyl ether and/or alkylaryl ether phosphate esters.

The amount of anionic surfactant, if used, is preferably between 0.5 and 5% by weight relative to the weight of the emulsion, advantageously between 0.5 and 2% by weight relative to the same reference.

More particularly, the weight ratio of emulsion relative to the external aqueous phase is between 30/70 and 90/10, preferably between 50/50 and 90/10.

According to one version of the invention the dispersion comprises at least one thickener. More particularly, said thickener is located in the continuous external aqueous phase.

The thickener is selected preferably from polysaccharides, such as xanthan gum and guar, alone or in mixtures.

Moreover, the amount of thickener is advantageously between 0.1 and 2% by weight of the dispersion (emulsion and continuous external aqueous phase).

On an ad hoc basis, it may be advantageous to incorporate at least one salt and/or sugar and/or polysaccharide into the continuous external aqueous phase. Thus, in the case in which the dispersion according to the invention (emulsion and external aqueous phase) is mixed with a multiple emulsion of water/oil/water type, it may be necessary to equilibrate the osmotic pressures of the external aqueous phase of the dispersion according to the invention (which will then be the same as the external aqueous phase of the multiple emulsion) and of the internal aqueous phase of the multiple emulsion.

Consequently, and if necessary, the continuous external aqueous phase of the dispersion according to the invention may comprise at least one salt selected from alkali metal or alkaline-earth metal halides, or alkali metal or alkaline-earth metal sulfates, or a mixture thereof, the concentration of salt in the continuous external aqueous phase being between 0.05 and 1 mol/l, preferably from 0.1 to 0.4 mol/l.

The continuous external aqueous phase may further comprise at least one sugar or at least one polysaccharide or mixtures thereof; the concentration of sugar and/or polysaccharide is such that the osmotic pressure of the continuous external aqueous phase comprising the sugar and/or polysaccharide corresponds to the osmotic pressure of a continuous external aqueous phase containing from 0.05 to 1 mol/l of salt.

The invention further provides a dispersion of the emulsion according to the invention in a continuous external organic phase.

More precisely, the emulsion according to the invention is dispersed in an external organic phase which is immiscible with the continuous phase of said emulsion.

As far as the nature of the continuous organic phase in which the emulsion according to the invention is dispersed is concerned it may be selected from the compounds listed above in the context of the description of the liquid L2 of the emulsion. This continuous external organic phase may likewise be of the same chemical nature as the disperse phase of the emulsion according to the invention, or not.

Furthermore, the continuous external organic phase comprises a stabilizer. Advantageously, this stabilizer is selected from comb or block copolymers of which one fraction is soluble in the disperse phase (in other words the continuous phase of the emulsion according to the invention) and the other in the continuous external organic phase, the fraction soluble in the continuous external organic phase being greater than the fraction soluble in the disperse phase.

Everything described above in relation to the copolymer employed to stabilize the emulsion according to the invention (L2/L1) will also apply in the case of the stabilizer stabilizing the emulsion dispersed in a continuous external organic phase, subject to the condition that the greater fraction of the polymer is that which is soluble in the continuous external organic phase, being different in nature from that which forms the continuous phase of the emulsion according to the invention.

Finally, the weight ratio of emulsion (L2/L1) relative to the continuous external organic phase is, in accordance with one version, between 30/70 and 90/10, preferably between 50/50 and 90/10.

The emulsion implemented in the method according to the invention can be obtained, for example., by preparing, on the one hand, a first mixture comprising the disperse organic phase, optionally comprising one or more active substances, and, on the other hand, a second mixture comprising the silicone and the polymer, and then in mixing the first mixture with the second, with stirring.

Where the continuous phase is of low viscosity (viscosity less than 1 Pa.s), stirring is preferably vigorous, and may advantageously be effected by the use of an apparatus of the Ultra-Turrax® or Microfluidizer type, or any high-pressure homogenizer.

Where the continuous phase is viscous (viscosity greater than or equal to 1 Pa.s, preferably greater than or equal to 5 Pa.s), agitation may advantageously be effected using a gate paddle.

The emulsion is generally prepared at a temperature of between 20 and 80° C.

The stirring time may be determined readily by the skilled worker, and depends on the type of apparatus employed. It is preferably sufficient to give an average droplet size of between 0.1 and 10 μm, preferably between 0.1 and 5 μm, (measured by means of a Horiba granulometer).

As regards the aqueous formulations stabilized by the method according to the invention, these formulations, as indicated above, contain up to 40% by weight of surfactant(s), preferably up to 25% by weight of surfactant(s). Moreover, the aqueous formulations contain at least 2% by weight of surfactant(s), more particularly at least 5% by weight of surfactant(s), and preferably at least 10% by weight of surfactant(s).

The amount of emulsion in the aqueous formulation is preferably such that the amount of disperse phase (oil/wax/fat and/or derivative), optionally combined with one active substance soluble in said disperse phase, in the cosmetic or dermatological formulation is between 0.01 and 50% by weight of the aqueous formulation.

These aqueous formulations are used more particularly in the field of the treatment of the skin and/or hair and in particular as shampoos for hair or for the body, body or facial cleansing gels, liquid soaps, foaming compositions for the bath, conditioners, formulations for styling hair and for making it easier to comb hair, among others.

Among surfactants forming part of the composition of such aqueous formulations, mention may be made, by way of anionic surfactants, of the following:

alkyl ester sulfonates, alkylbenzenesulfonates, primary or secondary alkylsulfonates, alkylglycerol sulfonates, sulfonated polycarboxylic acids, and paraffinsulfonates;

alkyl sulfates;

alkyl ether sulfates;

alkylamidesulfates;

salts of saturated or unsaturated fatty acids, N-acyl-N-alkyltaurates, alkylisethionates, alkylsuccinamates and alkylsulfosuccinates, sulfosuccinate monoesters or diesters, N-acylsarcosinates, and polyethoxycarboxylates; and

alkyl and/or alkyl ether and/or alkylaryl ether phosphate esters; alone or in a mixture.

Among the nonionic surfactants which can be used in the formulations, those which are suitable include the following:

alkoxylated fatty alcohols;

alkoxylated triglycerides;

alkoxylated fatty acids;

alkoxylated sorbitan esters;

alkoxylated fatty amines;

alkoxylated di(1-phenylethyl)phenols;

alkoxylated tri(1-phenylethyl)phenols;

alkoxylated alkylphenols;

products resulting from the condensation of ethylene oxide with a hydrophobic compound resulting from the condensation of propylene oxide with propylene glycol, such as the Pluronic products sold by BASF;

products resulting from the condensation of ethylene oxide the compound resulting from the condensation of propylene oxide with ethylenediamine, such as the Tetronic products sold by BASF;

alkylpolyglycosides such as those described in U.S. Pat. No. 4,565,647;

amides of fatty acids, for example C₈-C₂₀ acids; alone or in a mixture.

The formulations may likewise comprise cationic surfactants such as alkyldimethylammonium halides, or else amphoteric or zwitterionic surfactants, such as betaines, for example lauryl betaine (Mirataine BB from the company Rhodia Chimie); sulfobetaines; amidoalkylbetaines, such as cocamidopropylbetaine (Mirataine BDJ from the company Rhodia Chimie); sultaines such as cocamidopropylhydroxysultaine (Mirataine CBS from the company Rhodia Chimie); alkylamphoacetates and alkylamphodiacetates, such as those, for example, comprising a coco chain, lauryl (Miranol C2M, C32 in particular from the company Rhodia Chimie); alkylamphopropionates or alkylamphodipropionates (Miranol C2M SF); and alkylamphohydroxypropylsultaines (Miranol CS) alone or in combination.

It will be appreciated that a plurality of categories of surfactants may be present in a single formulation.

It is possible thus to incorporate into the cosmetic composition, in dispersion or solution form, bactericides or fungicides, for the purpose of improving skin disinfection, such as triclosan, for example; antidandruff agents, such as, in particular, zinc pyrithione or octopirox; and insecticides, such as natural or synthetic pyrethroids.

The aqueous formulations may also include agents for protecting the skin and/or hair against the aggressive effects of the sun and of UV rays. Accordingly the compositions may comprise solar filters, which are chemical compounds with high absorption for UV radiation, such as the compounds authorized in European Directive 76/768/EEC, its annexes and the subsequent amendments to said directive.

The aqueous formulations may also include fixative resins.

These fixative resins, when present, are generally present at concentrations of between 0.01 and 10%, preferably between 0.5 and 5%.

The fixative resins forming part of the aqueous formulations are more particularly selected from the following resins:

methyl acrylate/acrylamide copolymers, polyvinyl methyl ether/maleic anhydride copolymers, vinyl acetate/crotonic acid copolymers, octylacrylamide/methyl acrylate/butylaminoethyl methacrylate copolymers, polyvinylpyrrolidones, polyvinylpyrrolidone/methyl methacrylate copolymers, polyvinylpyrrolidone/vinyl acetate copolymers, polyvinyl alcohols, polyvinyl alcohol/crotonic acid copolymers, polyvinyl alcohol/maleic anhydride copolymers, hydroxypropylcelluloses, hydroxypropylguars, sodium polystyrenesulfonates, polyvinylpyrrolidone/ethyl methacrylate/methacrylic acid terpolymers, poly(methyl vinyl ether/maleic acid) monomethyl ethers, and polyvinyl acetates grafted onto polyoxyethylene backbones (EP 219048);

copolyesters derived from terephthalic and/or isophthalic and/or sulfoisophthalic acid, anhydride or a terephthalic and/or isophthalic and/or sulfoisophthalid diester, and a diol, such as:

polyester copolymers based on ethylene terephthalate and/or propylene terephthalate and polyoxyethylene terephthalate units (U.S. Pat. No. 3,959,230, U.S. Pat. No. 3,893,929, U.S. Pat. No. 4,116,896, U.S. Pat. No. 4,702,857, U.S. Pat. No. 4,770,666);

sulfonated polyester oligomers obtained by sulfonating an oligomer derived from ethoxylated allyl alcohol, dimethyl terephthalate and 1,2-propylenediol (U.S. Pat. No. 4,968,451);

polyester copolymers derived from dimethyl terephthalate, isophthalic acid, dimethyl sulfoisophthalate, and ethylene glycol (EP 540374);

copolymers comprising polyester units derived from dimethyl terephthalate, isophthalic acid, dimethyl sulfoisophthalates, and ethylene glycol and polyorganosiloxane units (FR 2728915);

sulfonated polyester oligomers obtained by condensing isophthalic acid, dimethyl sulfosuccinate, and diethylene glycol (FR 2236926);

polyester copolymers based on propylene terephthalate and polyoxyethylene terephthalate units and terminated by ethyl units, methyl units (U.S. Pat. No. 4,711,730), or polyester oligomers terminated by alkylpolyethoxy groups (U.S. Pat. No. 4,702,857) or anionic sulfopolyethoxy groups (U.S. Pat. No. 4,721,580) or sulfoaroyl groups (U.S. Pat. No. 4,877,896);

polyester-polyurethanes, more particularly those obtained by reacting a polyester obtained from adipic acid and/or terephthalic acid and/or sulfoisophthalic acid and a diol with a prepolymer containing terminal isocyanate groups, obtained from a polyoxyethylene glycol and a diisocyanate (FR 2334698);

ethoxylated monoamines or polyamines, and polymers of ethoxylated amines (U.S. Pat. No. 4,597,898, EP 11984).

Preferentially the fixative resins are selected from polyvinylpyrrolidone (PVP), copolymers of polyvinylpyrrolidone and methyl methacrylate, the copolymer of polyvinylpyrrolidone and vinyl acetate (VA), polyethylene glycol terephthalate/polyethylene glycol copolymers, polyethylene glycol terephthalate/polyethylene glycol/polysodium sulfonate isophthalate copolymers, and mixtures thereof.

The aqueous formulations may also include polymeric derivatives which exert a protective function.

These polymeric derivatives may be present in amounts of the order of 0.01-10%, preferably approximately 0.1-5%, and more particularly of the order of 0.2-3% by weight.

These derivatives may in particular be selected from:

nonionic cellulose derivatives such as cellulose hydroxyethers, methylcellulose, ethylcellulose, hydroxypropylmethylcellulose, and hydroxybutylmethylcellulose;

polyvinyl esters grafted onto polyalkylenated backbones, such as the polyvinyl acetates grafted onto polyoxyethylene backbones (EP 219048);

polyvinyl alcohols.

The aqueous formulations may also comprise plasticizers.

Said plasticizers, when present, represent between 0.1 to 20% of the formulation, preferably from 1 to 15%.

Among particularly useful plasticizers mention may be made of adipates, phthalates, isophthalates, azelates, stearates, silicone copolyols, glycols, castor oil, or mixtures thereof.

It is likewise possible to incorporate humectants into said aqueous formulations; humectants include, inter alia, glycerol, sorbitol, urea, collagen, gelatin, aloe vera, hyaluronic acid or volatile water-soluble solvents such as ethanol or propylene glycol, the amounts of which may reach up to 60% by weight of the composition.

In order further to reduce the irritation or aggression of the scalp it is also possible to add water-soluble or water-dispersible polymers such as collagen or certain non-allergenic derivatives of animal or plant proteins (wheat protein hydrolysates, for example), natural hydrocolloids (guar gum, carob gum, tara gum, etc.) or hydrocolloids obtained from fermentation processes, and derivatives of these polycarbohydrates, such as nonionic modified celluloses, hydroxyethylcellulose for example, or anionic modified celluloses, such as carboxymethylcellulose; guar derivatives or carob derivatives, such as their nonionic derivatives (hydroxypropylguar, for example) or the anionic derivatives (carboxymethylguar and carboxymethylhydroxypropylguar).

To these compounds it is possible to add, in combination, mineral particles or powders, such as calcium carbonate, sodium bicarbonate, calcium dihydrogenphosphate, mineral oxides in powder form or in colloidal form (particles with a size smaller than or of the order of one micrometer, sometimes several tens of nanometers), such as titanium dioxide, silica, aluminum salts, which are used generally as antiperspirants, kaolin, talc, clays and their derivatives, etc.

Preservatives, such as the methyl, ethyl, propyl and butyl esters of p-hydroxybenzoic acid, sodium benzoate, Germaben® or any chemical preventing the proliferation of bacteria or molds which is traditionally used in aqueous formulations may also be introduced into the stabilized aqueous formulations according to the invention, generally at a level of from 0.01 to 3% by weight.

The amount of these products is commonly adjusted in order to prevent any proliferation of bacteria, molds or yeasts in the aqueous formulations.

Antioxidants may optionally be incorporated into the aqueous formulations.

Alternatively to these chemicals it is sometimes possible to use agents which modify the activity of water and which greatly increase the osmotic pressure such as carbohydrates or salts.

In order to protect the skin and/or hair from attack by the sun and UV rays it is possible to add to these formulations mineral particles such as zinc oxide, titanium dioxide or cerium oxides, in powder form or in the form of colloidal particles, alone or in a mixture. These powders may optionally be surface-treated in order to enhance the effectiveness of their anti-UV activity or in order to facilitate their incorporation into the aqueous formulations or in order to inhibit the surface photoreactivity.

To these ingredients it is possible to add, if necessary, for the purpose of enhancing comfort when the composition is used by the consumer, one or more perfumes, colorants, among which mention may be made of the products described in annex IV (“List of coloring agents allowed for use in cosmetic products”) of European Directive 76/768/EEC of 27 Jul. 1976, known as the Cosmetics Directive, and/or opacifiers such as pigments.

Although not mandatory, the composition may also include viscosity-modifying or gelling polymers, in order to adjust the texture of the composition, such as crosslinked polyacrylates (Carbopol products, sold by Goodrich), noncationici cellulose derivatives such as hydroxypropylcellulose, carboxymethylcellulose, guars and their nonionic derivatives, xanthan gum and its derivatives, which are used alone or in combination, or the same compounds, generally in the form of water-soluble polymers modified with hydrophobic groups linked covalently to the polymer framework, as described in international application WO 92/16187, and/or water, in order to take the total of the constituents of the formulation to 100%.

The aqueous formulations may likewise include polymeric dispersants in an amount of the order of 0.1-7% by weight, in order to control the hardness in terms of calcium and magnesium; the dispersants are such as:

water-soluble salts of pplycarboxylic acids with a weight-average molecular mass of the order to 2000 to 100000 g/mol, obtained by polymerization or copolymerization of ethylenically unsaturated carboxylic acids such as acrylic acid, maleic acid or anhydride, fumaric acid, itaconic acid, aconitic acid, mesaconic acid, citraconic acid, methylenemalonic acid, and very particularly polyacrylates with a weight-average molecular mass of the order of 2000 to 10000 g/mol (U.S. Pat. No. 3,308,067), and copolymers of acrylic acid and maleic anhydride with a weight-average molecular mass of the order of 5000 to 75000 g/mol (EP 66915);

polyethylene glycols, for example with a weight-average molecular mass of the order of 1000 to 50000 g/mol.

They may also comprise metal sequestrants, more particularly calcium sequestrants, such as citrate ions, for example.

A specific example will now be presented.

### EXAMPLE

1/ Synthesis of a Polybutyl Acrylate 3000—Polydimethylsiloxane 35000—Polybutyl Acrylate 3000 Triblock Copolymer.

1a. Preparation of the Alpha, Omega-Bis (Xanthate) PDMS Oil

1 molar equivalent of alpha,omega-carbinol silicone oil containing 422 dimethylsiloxane units (¹H NMR analysis) is dissolved in 500 ml of cyclohexane in the presence of 4 molar equivalents of triethylamine, NEt₃.

2.4 equivalents of 2-bromopropionyl bromide in solution in 100 ml of cyclohexane are introduced via a cannula.

A white precipitate is formed immediately, with a slight increase in the temperature of the reaction mixture.

The aqueous phase is subsequently separated from the organic phase. The cyclohexane is then evaporated.

The crude product obtained is subsequently iltered over a bed of silica. The solvents are then vaporated so that the acylation product is recovered.

One equivalent of the product obtained above is added at ambient temperature to a solution of 4 equivalents of xanthate salt (K⁺, ⁻SCSOEt) in acetonitrile. The mixture is stirred at ambient temperature for 4 hours.

The solvent is then evaporated under reduced pressure and the resulting solid is mixed with ether and filtered on a frit. The solvent is subsequently evaporated.

1b. Synthesis of Polybutyl Acrylate 3000—Polydimethylsiloxane 35000—Polybutyl Acrylate 3000 Triblock Copolymer

0.05 g of azobisisobutyronitrile are introduced into a glass reactor simultaneously with 20 g of the PDMS oil described in example 1a, 3.43 g of butyl acrylate and 70.3 g of cyclohexane.

The mixture is heated at 70° C. for 7 hours.

The solvent is then evaporated.

2/ Preparation of the Ethanol/Silicone Oil (80/20) Emulsion

First of all, 1 g of the copolymer obtained above is added to 1 g of silicone oil (Rhodorsil 47V1000; sold by Rhodia Chimie). The mixture is then brought to 5° C. and homogenized at 400 rpm using a solid gate paddle.

8 g of a resin solution in ethanol (Resyn 28-2930, sold by National Starch; 40% resin solution neutralized to 90% by adding 95% 2-amino-2-methyl-1-propanol, Aldrich) are added dropwise to the silicone phase. At the end of addition, shearing is maintained at 400 rpm for 20 minutes, for refining.

A stable emulsion is obtained.

3/Preparation of the Dispersion of the Emulsion

An aqueous phase is prepared into which the emulsion will be dispersed, comprising 16% of Empicol ESB3M (sodium laureth sulfate, sold by Rhodia Chimie); 2% of Tegobetaine 7 (cocamidopropyl betaine, sold by Goldschmidt) and 2% of Miranol Ultra C32 (cocoamphodiacetate, sold by Rhodia Chimie).

0.5 g of multiple emulsion obtained in section 2/ is poured into approximately 2 g of the aqueous phase prepared above, and the combined mixture is stirred manually.

It is observed that the ethanol/silicone/water multiple emulsion is stable in this surfactant-rich medium, as shown by the figure, which is an optical microscope view of the diluted emulsion.

1-11. (Canceled)

12. A method of stabilizing an aqueous formulation containing up to 40% by weight of one or more surfactants and comprising at least one organic phase being organic or inorganic oils, organic or inorganic fats, organic or inorganic waxes, products obtained from the alcoholysis of these oils, fats, waxes, essential oils, mono-, di- and tri-glycerides, fatty acids, esters of such acids, or alcohols, said method comprising the step of adding to said aqueous formulation an emulsion having a continuous and disperse phase:
whose continuous phase is at least one silicone, the disperse phase being the aforementioned organic phase; and
which is stabilized by at least one comb or block copolymer of which one fraction is soluble in the disperse phase and the other in the continuous phase, the fraction soluble in the continuous phase being greater than the fraction soluble in the disperse phase.

13. The method according to claim 12, wherein the silicone is composed in whole or in part of units of formula:
R′₃₋ₐRₐSiO_{1/2} (unit M) and/or R₂SiO (unit D)
wherein:
a is an integer from 0 to 3;
the radicals R are identical or different and represent:
a saturated or unsaturated aliphatic hydrocarbon group containing 1 to 10 carbon atoms;
an aromatic hydrocarbon group containing 6 to 13 carbon atoms; or
a polar organic group bonded to the silicon by an Si—C or Si—O—C bond;
the radicals R′ are identical or different and represent:
a saturated or unsaturated aliphatic hydrocarbon group containing 1 to 10 carbon atoms;
an aromatic hydrocarbon group containing 6 to 13 carbon atoms;
an —OH function; or
an amino- or amido-functional group containing 1 to 6 carbon atoms, bonded to the silicon by an Si—N bond.

14. The method according to claim 12, wherein the disperse phase is in a proportion by weight relative to the continuous phase of between 10/90 and 90/10.

15. The method according to claim 14, wherein proportion is of between 30/70 and 50/50.

16. The method according to claim 12, wherein the copolymer is a copolymer comprising at least three blocks.

17. The method according to claim 12, wherein the copolymer is in an amount of from 0.5 to 10% by weight of the disperse phase.

18. The method according to claim 12, wherein the disperse phase further comprises at least one active substance soluble in said disperse phase.

19. The method according to claim 12, wherein the formulation is for the treatment of the skin or hair.

20. The method according claim 19, wherein the formulation is a shampoo formulation for the hair or for the body, a cleansing gel formulation for the face or body, a liquid soap formulation, a foaming composition formulation for the bath, a conditioner formulation, a styling formulation or a formulation for making it easier to comb the hair.

21. The method according to claim 12, wherein the aqueous formulation contains up to 25% by weight of surfactant(s).

22. The method according to claim 12, wherein the aqueous formulation further contains at least 2% by weight of surfactant(s).

23. The method according to claim 22, wherein the aqueous formulation contains at least 10% by weight of surfactant(s).

24. The method according to claim 12, wherein the aqueous formulation presents an emulsion content such that the amount of disperse phase, optionally in combination with an active substance which is soluble in said disperse phase, is between 0.01 and 50% by weight of the aqueous formulation.
